(19) United States
(12) Patent Application Publication
(10) Pub. No.: US 2006/0247660 A1
(43) Pub. Date: Nov. 2, 2006

(54) Corneal retention or stabilizing tool

(76) Inventor: Edward Perez, Palo Alto, CA (US)

Correspondence Address: E. THOMAS WHEELOCK, P.O. BOX 61168, PALO ALTO, CA, 94306, US

(21) Appl. No.: 11/386,231

(22) Filed: Mar. 21, 2006

Related U.S. Application Data
(63) Continuation of application No. PCT/US04/31231, filed on Sep. 22, 2004.
(60) Provisional application No. 60/505,219, filed on Sep. 22, 2003.

(51) Int. Cl. A61F 9/00

(52) U.S. Cl. 606/107

(57) ABSTRACT

Described here is a surgical device that typically is used to releasably hold the cornea of a human eye (and hence that eye) in such a way as to modestly deform the cornea and the eye, to maintain the eye's position for procedures upon the epithilial layer of the cornea, and to allow ease of replacement of an epithilial flap should one be produced. It may be used in combination with an epithilial delaminating tool, or an ocular device inserting tool. The stabilization device permits ready access to and creation of flaps or pockets of epithelium for later introduction of correcting lenses (e.g. using an ocular device insertion tool) or subtractive procedures such as LASIK or LASEK, prior to replacement of epithelium over the corrective lens or over the site of laser induced or surgically-induced corrective procedure.

### FIELD OF THE DISCLOSURE

Described here is a surgical device that typically is used to releasably grasp and to hold the cornea of a human eye (and hence that eye) in such a way as to modestly deform or extend the cornea and the eye, to maintain the eye's position for procedures upon the epithilial layer of the cornea, and to allow ease of replacement of an epithilial flap should one be produced. It may be used in combination with an epithilial delaminating tool, or an ocular device inserting tool. The stabilization device permits ready access to and creation of flaps or pockets of epithelium for later introduction of correcting lenses (e.g. using an ocular device insertion tool) or subtractive procedures such as LASIK or LASEK, prior to replacement of epithelium over the corrective lens or over the site of laser induced or surgically-induced corrective procedure.

### BACKGROUND

Refractive surgery refers to a set of surgical procedures that change the native optical or focusing power of the eye. These changes alleviate the need for glasses or contact lenses that an individual might otherwise be dependent on for clear sight. The majority of the focusing power in the human eye is dictated by the curvature of the air-liquid interface, where there is the greatest change in the index of refraction. This curved interface is the outer surface of the cornea. The refractive power of this interface accounts for approximately 70% of the total magnification of the eye. Light rays that make up the images we see pass through the cornea, the anterior chamber, the crystalline lens, and the vitreous humor before they are focused on the retina to form an image. It is the magnifying power of this curved, air-corneal interface that provided the field of refractive surgery with the opportunity to surgically correct visual deficiencies.

A largely flawed and failed procedure called epikeratophakia was developed in the era of RK. It is now essentially an academic anomaly. Epikeratophakia provided a new curvature to the outer curvature of the cornea by grafting onto the cornea a thin layer of preserved corneal tissue.

The epikeratophakia lens was placed into the eye surgically. An annular 360° incision was made in the cornea after completely removing the epithelium from the epikeratophakic lens site. The perimeter of this lens would be inserted into the annular incision and held in place by a running suture. There were several problems with epikeratophakia: 1) the lenses remained cloudy until host stromal fibroblasts colonized the lens, which colonization possibly could take several months; 2) until migrating epithelium could grow over the incision site onto the surface of the lens, the interrupted epithelium was a nidus for infection; and 3) epithelium healing onto the surgical site sometimes moved into the space between the lens and the host cornea. Currently, epikeratophakia is limited in its use. It is now used in pediatric aphakic patients who are unable to tolerate very steep contact lenses.

Major industrial research efforts tried to produce a synthetic version of the epikeratophakic graft called the synthetic onlay in a synthetic epilens. Different synthetic polymers were used (hydroxyethylmethacrylate, polyethylene oxide, Lidofilcon, polyvinyl alcohol). Hydrogels of these materials normally do not have a surface readily conducive to the growth and adherence of epithelial cells onto their synthetic surfaces. This was one of the major setbacks of synthetic onlays. Epithelial cells could not adequately heal onto these lenses.

Another problem with these synthetic lenses is that they did not adhere well to the surface of the eye. Conventional suturing is difficult and the use of biological glues remains limited. Glues were not ideally biocompatible in the cornea.

Lastly, the permeability of these hydrogels was significantly limiting. Living epithelial cells on the surface had difficulty achieving adequate nutrition. Corneal epithelial nutritional flow flows from the aqueous humor, through the cornea, and out to the epithelial cells. In sum, industrial efforts to then had failed to develop an adequate synthetic epikeratophakic lens.

Around the mid 1990's, procedures that sculpt the cornea with lasers were sufficiently successful that they began to replace radial keratotomy. The first generation of laser ablation of the cornea was called photorefractive keratectomy (PRK). In PRK, an ablative laser (e.g., an excimer laser) is focused on the cornea to sculpt a new curvature into the surface. In PRK, the epithelium is destroyed when achieving a new outer surface curve. Over the ensuing post-operative days, the epithelium either grows or heals back into place. This epithelial healing phase was problematic for most patients since the epithelially denuded and ablated cornea was painful. The patient's vision is initially poor; this “recuperative time” can last from days to a week or more.

A subsequent variation of PRK corneal laser ablation, LASIK, has become very popular. The LASIK procedure, also known as LAser In Situ Keratomileusis, is synonymous in the public mind with laser vision correction. In LASIK, an outer portion (or chord-like lens-shaped portion) of the cornea (80 to 150 microns thick) is surgically cut from the corneal surface. This step is performed using a device called a microkeratome. The microkeratome cuts a circular flap from the surface of the cornea which flap containing both corneal tissue and epithelium remains hinged at one edge. This flap is reflected (or folded) back and an ablative (excimer) laser is used to remove or to reform a portion of the exposed surgical bed. The corneal flap is laid back into place. When this flap is laid back into place, the cornea achieves a new curvature because the flap conforms to the laser-modified surface. In this procedure, epithelial cells are not removed or harmed. The epithelial cells have simply been incised at the edge of this flap. When the flap is placed back onto the corneal bed, the epithelium heals back at the incision site. There is essentially no recuperative time and the results are almost immediate. Because there is very little surgical time (15 minutes for each eye) and because there are lasting and very accurate results, LASIK is currently considered the premier manner of performing refractive surgery.

The newest technique being evaluated in high volume refractive surgical practices and in some academic centers is a procedure called Laser Assisted Subepithelial Keratomileusis (LASEK). In LASEK, a “flap” is made of only epithelium. This layer of epithelium is contacted with an ethanol solution and is lifted off the cornea in a manner similar to LASIK. The ablative laser is focused just on the surface of the denuded cornea (in the same manner as was done with PRK). However, this epithelial flap is left physically intact, i.e., epithelium is not destroyed, but the epithelial cells are not truel viable. See, e.g., Investigative Ophthalnaology & Visual Science, by Chen et al. (Vol 43: 8 pp. 2593-2602, August 2002). It is simply rolled back into place after formation of the re-curved anterior portion of the cornea, resulting in much less recuperative time than with PRK. Current methods of LASEK are not as good as LASIK but the results are better than with PRK.

The corneal epithelium is a multilayered epithelial structure typically about 50 μm in thickness. It is non-cornified. The outer cells are living, although they are squamous in nature. The basal epithelial cells are cuboidal and sit on the stromal surface on a structure known as Bowman's membrane. The basal cell layer is typically about 1 mil thick (0.001″). The basal cells produce the same keratins that are produced in the integument, i.e., skin. The basal epithelial cells express keratins 5 and 14 and have the potential to differentiate into the squamous epithelial cells of the corneal epithelium that produce keratins 6 and 9. The corneal epithelium has a number of important properties: 1) it is clear; 2) it is impermeable; 3) it is a barrier to external agents; and 4) it is a highly innervated organ. Nerves from the cornea directly feed into the epithelium, and thus, defects of this organ produce pain.

Epithelial cells are attached side-to-side by transmembrane molecules called desmosomes. Another transmembrane protein, the hemidesmosome, connects to collagen type 7 and is present on the basolateral surface of basal epithelial cells. Hemidesmosomes anchor epithelium to the underlying collagenous portion of the stroma. The junction between the epithelium and corneal stroma is referred to as basement membrane zone (BMZ).

When a LASEK procedure is performed, a physical well is placed or formed on the epithelium and filled with a selection of 20 percent ethanol and balanced salt solution. Contact with the solution causes the epithelial cells to lose their adherence at the BMZ, most likely by destroying a portion of that cell population. The epithelium is then raised by pushing the epithelium, e.g., with a Weck sponge, in a manner similar to stripping a wall of paint. The exposed collagenous portion of the corneal stroma is then ablated to reshape its surface. A weakened epithelium is then rolled back into place to serve as a bandage. However, this “bandage” is not an epithelium in its original state. The epithelium is not in its original integral state, thereby reducing its clarity, impermeability to water, and barrier function. Furthermore, the ability of the epithelium to adhere to the corneal stromal surface is impaired.

U.S. Pat. Nos. 6,099,541 and 6,030,398 to Klopotek describe an microkeratome apparatus and method for cutting a layer of corneal epithelium to prepare the eye for LASIK or other reshaping procedures. The epithelium, if replaced, is attached using surgical techniques.

None of the cited references shows or suggests the device and procedures described herein.

### SUMMARY

Described here is a vacuum stabilization device for grasping, stabilizing, and modestly deforming the front portion of a human cornea (hereinafter referred to as a “stabilization device” or “stabilizer”). The stabilization device allows access to the cornea with a supplemental device such as an epithelial delaminator, ocular device inserter, or a laser device. The device may also guide, assist, or index the movement of these devices and others with respect to the cornea.

Generally, the described stabilization device includes an annular ring having at least two surfaces that contact the surface of the eye and define an annular region. An outer radial surface is situated or configured so that it typically first contacts the surface of the eye when the stabilizing device is introduced to the eye surface. In many variations of the depicted device, the inner radial surface defining the annular vacuum area typically does not contact the eye until the cornea and the eye itself is slightly deformed. In some versions, the inner radial surface defining the annular vacuum area does not contact the eye until the stabilization device is slightly deformed, or a until both the eye and the stabilization device are slightly deformed. The device includes an opening, typically circular, that is interior to the inner radial surface and hence not under the influence of the vacuum, and provides a stable region for, e.g., epithelium lifting.

The open central area may, in some variations, couple with a separate component—“a vacuum former”—to allow or to cause a vacuum that is imposed upon the annular region also to communicate with that circular area and to change the shape of the eye and cause the cornea to modestly protrude from that open central area. The “vacuum former” may be a separate component or may be the operating physician's thumb or the like. In some versions, the vacuum is formed by merely pressing the stabilizing device down onto the eye.

The corneal stabilizer may be used in conjunction with epithilial delaminators as may be found in published international application WO 03/061518, published Jul. 31, 2003, the entirety of such document is incorporated by reference. The corneal stabilizer may be used in conjunction with an ocular device inserter as may be found in a U.S. patent application Ser. No. 10/937,206, entitled OCULAR DEVICE APPLICATOR by Perez (filed Sep. 8, 2004) and a U.S. patent application Ser. No. 10/937,135, entitled COMBINED EPITHELIAL DELAMINATOR AND INSERTER, by Perez et al. (filed Sep. 8, 2004); the entirety of these documents are herein incorporated by reference.

The stabilization device may also include an index, or guide, configured to provide support, guide, assist, or index the movement of these devices and others with respect to the cornea, and with respect to the stabilization device. In one version, the guide is a track. In one version, the guide is configured to couple to another device (e.g. an epithelial delaminator, inserter, etc.).

Also included are kits of apparatus, i.e., the disclosed stabilization device in combination with an epithilial delaminator configured to completely remove a section of epithelium, an epithilial delaminator configured to remove a flap of epithelium, or an epithilial delaminator configured to produce a limited flap or pocket of epithelium with one or more openings. The kits including this disclosed and described corneal stabilizer may also include a vacuum maker suitable for closing the opening that otherwise provides access to the front of the cornea.

Also described here are procedures for use of the described corneal stabilizer including the steps of: (a.) providing a described corneal stabilizer, (b.) placing the described corneal stabilizer on an eye in an appropriate region of the eye, e.g., generally centered about the eye's cornea, (c.) providing a vacuum to the annular space of the stabilizer, (d.) optionally closing the open region at the front of the stabilizer to distribute the vacuum and to deform the eye (or simply pressing down on the stabilizer), to cause the eye to contact the inner radial surface of the stabilizer and seal the annular space in the stabilizer to the vacuum, thereby affixing the stabilizer to the eye, (e.) providing an epithelial delaminator, (f.) separating at least a portion of the epithelium from the cornea, and (g.) carrying out a procedure for correcting optical characteristics of the eye. The final several steps are optional to this described procedure. The procedure may include a variety of laser- or tool-induced corrective procedures or may include the simple step of placing a contact lens of some type on the de-epithelialized corneal surface. In each instance, it is desirable that the epithelium be replaced over the surgically altered site or the contact lens.

### BRIEF DESCRIPTION OF THE DRAWINGS

FIG. 1A is a perspective view of a typical corneal stabilizing device and the associated vacuum maker.

FIG. 1B shows a cross sectional view of the stabilizer found in FIG. 1A.

FIG. 2A shows another variation of the stabilizer.

FIG. 2B shows a cross sectional view of the stabilizer ring found in FIG. 2A.

FIG. 3A shows another variation of the stabilizer.

FIG. 3B shows a cross sectional view of the stabilizer ring found in FIG. 3A.

FIGS. 4A and 4B show surfaces that would be appropriate for the outer radial surface of the described stabilizer.

FIGS. 4C and 4D show surfaces that would be appropriate for the inner radial surface of the described stabilizer.

FIGS. 4E and 4F show cross-sections through variations of the outer radial surface of the described stabilizer.

FIGS. 5A to 5D a typical procedure for use of the stabilizer.

FIGS. 6A and 6B show another version of a procedure for use of the stabilizer.

FIGS. 7A and 7B show some delaminating devices that may be used variously in conjunction with the described stabilizer or as a portion of the kit.

FIG. 8 shows a cross-sectional view of another version of the stabilizer.

FIGS. 9A and 9B show cross-sectional views of other versions of the stabilizer.

### DETAILED DESCRIPTION

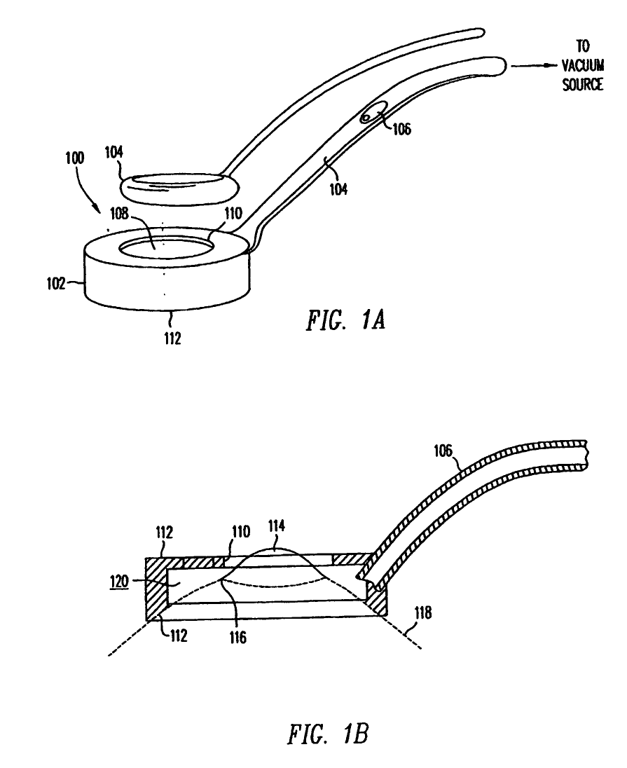

FIG. 1A shows a perspective view of one variation (100) comprising a base (102) and a vacuum former component (104). The base itself includes a line (104) to a vacuum source. Vacuum line (104) may be used as a handle and typically would be provided with a vacuum breaker orifice (106) allowing the user to manipulate or break the vacuum to the device when a removal is desired. The base section (102) has an opening (108) through which the human cornea projects after the device is completely deployed on the eye. As may be better seen in later figures, base (102) has an inner radial surface (110) that is configured to contact the eye during operation and to provide a seal for the vacuum and an outer radial surface (112) that also contacts the eye. The device may be automated, e.g. the control of the vacuum may be automatically controlled.

FIG. 1B shows the base member (102) in cross section. The outline of an eye is shown in outline simply with a cornea (114), a limbus (116), and sclera (118) for clarity of explanation. The base (102) is shown with the outer radial surface (112) in the position in which the disclosed stabilizer first contacts the eye. The inner radial surface (110) is also shown but it should be understood that, at this point, the inner radial surface (110) does not contact the eye or, at least does not seal against the eye and form a seal or distribute any supplied vacuum. The device is configured so that when a vacuum is applied through vacuum line (106) into the substantially annular chamber (120) and the opening (108) is closed, the anterior portion of the cornea is pulled up into contact with the inner radial surface (110). The relative size and placement of these two surfaces (110 and 112) provides for revision of the shape of the eye in a gross and temporary sense, and causes the movement of the cornea towards the open front opening (108), and fixes the device in position (102) with respect to the eye. These modest alterations of the corneal shape provide a surface of the cornea that is proud of (or extends from) the front of the stabilizer device (112). This is an easy surface upon which to perform a procedure.

Although, I do not wish to be bound by the range of these values, ranges of vacuums which are suitable for operable in this described device include: vacuum values of up to about 300 mm. Hg. and values in the neighborhood of 150 mm. Hg. Also, values in the range of 100 to 250 mm. of Hg. and 125 to 175 mm. of Hg. are suitable. As may be readily understood, the higher the vacuum value applied, the firmer the front surface of the cornea becomes.

Additionally, I have found that a distance between the inner radial surface and the corneal surface of about 1/16 of an inch is appropriate in these devices. That is to say that a suitable gap between the inner radial surface (110) and the cornea prior to the time that vacuum is applied may be 0.0625 inches+/−0.03 inches.

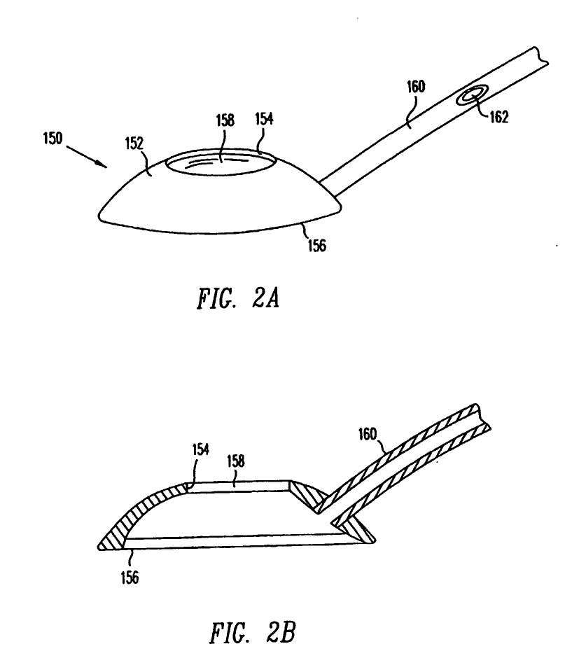

FIG. 2A shows another variation of the inventive stabilizer (150). In this case, the base member (152) does not provide as much of an open annular vacuum volume as does the variation shown in FIGS. 1A and 1B. Nevertheless, the components are substantially the same. That is to say, that the variation has a base member (150), an inner radial surface (154), an outer radial surface (156), an opening (158) for accessing the anterior corneal surface of an eye, a vacuum line (160), and a vacuum breaker (162). As noted just above, the presence of some volume of open vacuum volume between the inner radial surface (154) and the outer radial surface (156) is desirable.

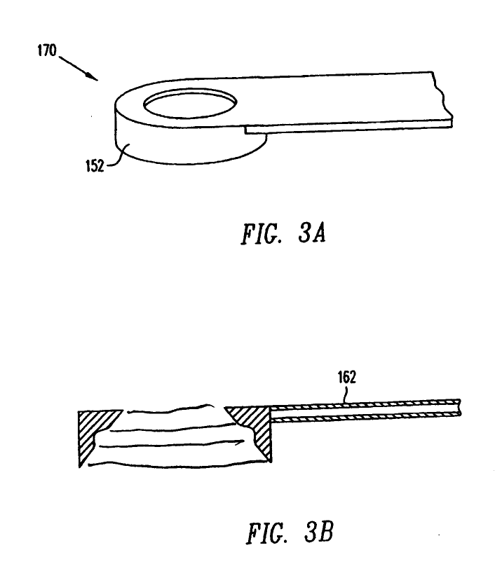

FIGS. 3A and 3B show another variation of stabilizer (170). In this version, the base member (152) is attached to a positioning member (172). The vacuum line (162) may apply a vacuum between the inner radial surface and an outer radial surface, as previously described. The positioning member may be connected to a holder, or an automatic positioner. In some versions, the positioning member is configured as a handle. In some versions of the stabilizer, there is no positioning member, and the stabilizer merely connects to a vacuum line.

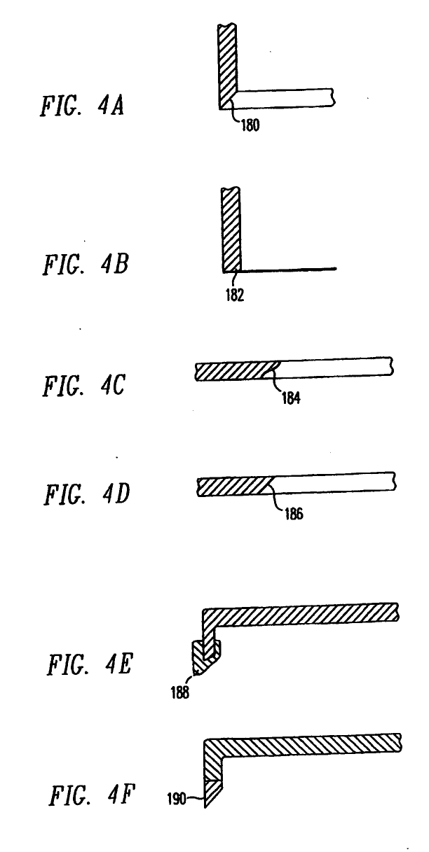

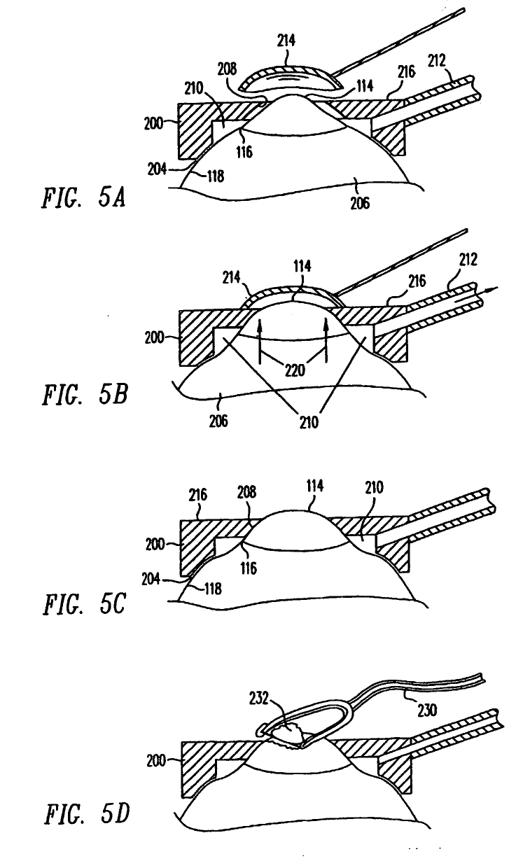

FIGS. 4A and 4B show suitable outer radial surfaces. The shape of the outer radial surfaces provided in FIGS. 1B and 2B, as well as in FIGS. 5A to 5D are acceptable. The surface (180) shown in FIG. 4A may be straight or slightly curved to cooperate with the shape of the eye, where contacted. The contact surface of (182) shown in FIG. 4B is a simple corner and is also acceptable although obviously providing an opportunity for more pronounced trauma to the eye.

FIG. 4C shows a variation of the inner radial surface (184) in which the surface is extended upwardly to provide a wider support region than would be otherwise available simply by machining a cornea-conforming shape from the material of manufacture. Inner radial surface shape (184) may be desirable in certain instances in which due to, e.g., significant astigmatism, the cornea is oddly shaped.

FIG. 4D shows an outer radial surface (186) that is simply a 45° cut.

In some versions, the surfaces of the device which contact the eye comprise a layer (e.g. a coating) to prevent damage to the eye. For example, the inner radial surface and the outer radial surface may be polished to prevent damage to the eye surface. In some versions, the inner and outer radial surfaces comprise a coating. For example, the inner and outer radial surfaces that contact the eye may be coated with a friction-reducing material, or a lubricant. In another version, the inner and outer radial surfaces may include a fluid, gel, or gel-like material (such as HA) that aids the formation or sealing of the vacuum.

FIGS. 4E and 4F show outer radial surfaces in which the region that contacts the eye (188 and 190) comprises a flexible material. In FIG. 4E, the flexible material is shown as a gasket on the stabilizer that comprises the outer radial surface (188). In FIG. 4F, a flexible material comprises an integral part of the stabilizer to comprise the outer radial surface (190). One variation of the described procedure is shown in FIG. 5A-5D. In FIG. 5A, base member (200) comprises a substantially ring-shaped member having an outer radial surface (204) configured to contact and form a seal with an eye (206) and an inner radial surface (208) configured to contact the eye to (206) after the device (204) has been deployed.

Generally the two surfaces, outer radial surface (204) and inner radial surface (208), are chosen in size and placement within the base member (200) such that when the base member (202) first contacts eye (206), the outer radial surface (204) it is in contact with the eye and inner radial surface (208) is not. The outer radial surface (204) is configured to contact the eye upon the sclera (118) although this is not a requirement of this device. The outer radial surface (204) may contact the eye in or on the limbus (116) or, in certain circumstances, upon the cornea (114) itself. In any case, during that initial step, the inner radial surface (208) is sized and positioned, both radially and anteriorally with respect to the outer radial surface (204), that it does not contact the eye or does not contact it in such a way as to form a seal with the cornea. After the device has been fully deployed, and the shape of the eye has been reformed, the inner radial surface (208) then contacts the eye and helps to form the annular vacuum volume. The inner radial surface (208) may contact some portion of the cornea, however, prior to deployment.

Inner radial surface (208) is configured so that it will contact the surface of the cornea after the desired temporary reformation of the cornea has been accomplished and to form a seal with the cornea. The annular vacuum volume (210) is defined as being between the inner radial surface (208) and the outer radial surface (204). The vacuum is introduced into the annular vacuum volume (210) by vacuum line (212) which, is noted above, may also serve as a device handle.

The configuration described above with respect to base member (200), particularly the spatial relationship between amongst the otuer radial surface (204), the inner radial surface (208), and the contact region of the cornea where the inner radial surface is to seal, is functionally the same in the event a vacuum former (214) is utilized to initiate the sealing of annular vacuum volume (210) or if the base member (200) is merely pressed into the eye to deform the eye slightly, but significantly, to cause the cornea to contact the inner radial surface (208) and seal the annular vacuum volume (210).

The kit including the base member (200) and a component (214) is a variation of the described material.

FIGS. 5A to 5D shows an extended version of one variation of my procedure. FIG. 5A shows the first step of placing the base member (200) on an eye (206). Note that the outer radial surface (204) contacts the eye and yet the inner radial surface (208) has not yet had contact with the eye. The vacuum making component (214) is shown approaching the front surface (216) of the base (200).

FIG. 5B shows the contact of vacuum making member (214) with the front surface of base (200) after application of vacuum through line (212). It is to be noted that closing of this system volume by application of vacuum into annular volume (210) causes the eye (206) and in particular the cornea (114) to move forward into contact with the inner radial surface (208) as shown by movement arrows (220).

FIG. 5C shows the complete deployment of the described device and presentation of a portion of the anterior corneal surface (114) through the open central area for a procedure. The anterior surface of the cornea stands proud of the front surface (216) of the device base (200). It should be noted that the device may also be sized in such a way that the inner radial surface (218) contacts the eye at the limbus (116) or even down upon the sclera (118).

In any case, the inner radial surface (208) and outer radial surface (204) in this variation form an annular vacuum volume (210) that, in combination, fix the base member (200) with respect to the eye (206) in such a way to stabilize the eye; that is to say, to prevent relative motion of the eye with respect to a later procedure performed on the epithilium, to slightly reform the eye, and to provide a measure of stiffness to the anterior portion of the cornea (114) upon which the procedure has had.

FIG. 5D shows the deployed device and the base member (200) in contact with the eye as described with reference to FIG. 5C just above. In this step, an epithelial delaminator (230) is shown to be removing epithelium from the surface of the cornea and form an epithilial flap (232).

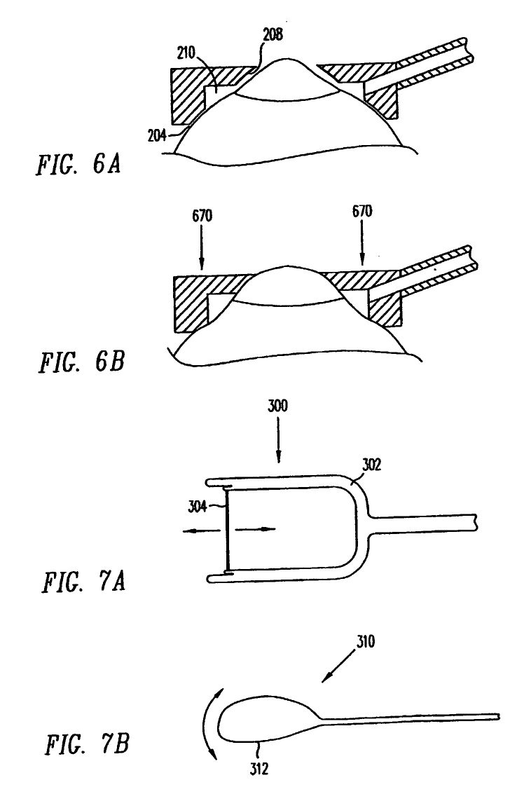

Another version of the procedure described herein for applying the stabilizer to the corneal surface is shown in FIGS. 6A and 6B. FIGS. 6A and 6B show that the application of pressure (or force) down on the stabilizer may be adequate to form a vacuum in the annular vacuum volume, and therefore secure the stabilizer onto the eye.

The step of delaminating the epithilial layer (232) from the corneal surface may entail any of the following variations. The epithelium may be simply separated from the cornea. It may be lifted from the corneal surface. The separation or lifting may further include removal of the separated epithelium from the cornea or it may entail the making of a flap having a hinge area in which the epithelium may be of a form that can rotate around the hinge with respect to the front of the cornea. It may be the forming of a simple pocket in which the only apparent and only slightly visible manifestations of the epithelial separation are the openings (or opening) into the pocket. In some variations of the then-following procedures, a lens may be introduced into the pocket. A device, perhaps with optical qualities, and perhaps not, may be introduced into the pocket or beneath the flap. It is likely that due to the nature of the devices used to measure the optic capabilities of the cornea and associated lens, that removal of the described stabilizer is desired prior to so called “subtractive” procedures used to correct vision. Such procedures include LASIK and LASEK.

FIG. 7A shows, for summary purposes only, an epithelial delaminating device (300) having a yoke (302) and a wire (304). The wire (304) provides a mechanism by which the epithelium may be mechanically separated from the cornea. The wire (304) may be vibrated in some fashion.

The vacuum stabilization device described may further comprise a guide or indexing platform to assist or to restrict (or to provide an indexed direction for) the movement of supplemental devices (such as an epithelial delaminator or ocular device inserter) when using the stabilization device on an eye. By “indexing,” I mean providing a stable set of coordinates amonth the described stabilization device, the eye, and any supplemental devices. In one version, the guide is a track configured to communicate with a portion of the supplemental device. For example, the vacuum device may comprise a slotted track located on a portion of the outer surface of the base region (102). The guide may be integral to the base region, or may project from the base region. In one version, a supplemental device (e.g. delaminator) comprises pins which fit into the stabilizer's track region, and guide the motion of the supplemental device relative to the eye. In one version, the movement of the supplemental device along the pathway may be regulated by the stabilization device. For example, the guide (e.g. track) may determine the “angle of approach” of a delaminator, as well as the angle from which the dealmination may occur. In some versions, the guide may be adjustable by the user, or automatically adjustable.

Additional supplemental devices (e.g. an inserter) may use the same, or a different, guide on a single stabilization device. Thus, an eye may be delaminated in a controlled manner, the dealaminator may be removed, and an inserter may be used to apply an ocular device beneath the dealaminated epithelium following the same pathway of the delaminator in the same eye. In one version, the stabilization device comprises separate guides for a dealminator and an inserter.

FIG. 7A shows a varation of an epithelial delamining device which may be used with the stabilizers described. FIG. 7B shows another variation of an epithelial delaminating device (310) having a vibrating or swinging wire (312) that may be used to make pockets beneath the epithelium. Examples of suitable epithelial delaminating devices are described in published PCT application WO 03/061518, the entirety of which is incorporated by reference.

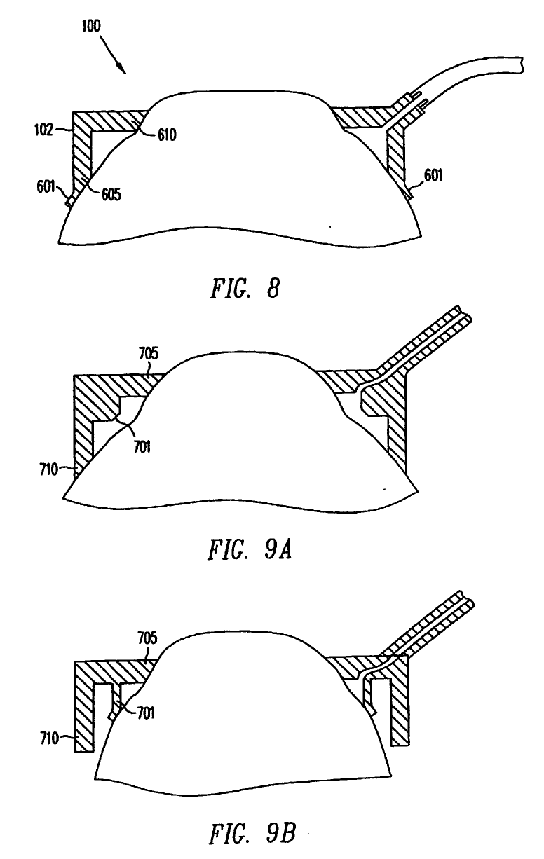

As described above, the stabilization device may also be configured to conform to a variety of different eye shapes or sizes. In some versions, the base region (102) further comprises a conformable “skirt.” FIG. 8 shows one version of the device in which at least one of the annular eye-contacting surfaces (shown as the outer radial surface (605)) includes a skirt region (610) which is sufficiently flexible to conform to the surface of the eye, particular under the force of an applied annular vacuum. The “skirt” region may allow a greater contact surface between the device and the eye, helping to prevent loss of the vacuum. Furthermore, the flexible skirt allows the device to adapt to a greater range of eye shapes (e.g. irregularly shaped eyes) or sizes. Although the outer radial surface is shown having a skirt, the inner radial region (610) may also comprise a conformable skirt.

The conformable skirt may comprise any material sufficiently pliable to fit onto the surface of the eye, yet capable of maintaining the vacuum within the device. Examples of materials include elastomeric materials, rubbers, soft polymers, and the like. In one version, the skirt is integral to the annular region (e.g. the inner annular region or outer annular region). In one version, the entire annular region may act as a “skirt,” at least partly conforming to the surface of the eye under an applied vacuum.

The stabilization device may also comprise more than one “outer” annular region to allow the device to be used with a broad variety of eye sizes. FIGS. 9A and 9B show alternative views of the device in which one additional, “middle” annular region (701) is included between an inner annular region (705) and an outer annular region (710). The middle annular region may allow the device to conform to narrower (or smaller) eyes for which the outer annular region would be to large. With larger (or broader) eyes, the middle annular region does not initially contact the eye, as shown in FIG. 9A. In some versions, the middle annular region may also help support the device when a vacuum is applied. FIG. 9B shows another version of a device having a middle annular region (701) which also comprises a flexible skirt. FIG. 9B shows the device under a vacuum, in which the middle annular region has sealed around the eye and the skirt region has conformed to part of the eye surface.

In addition, the described device may be included in a system of kit. In particularly, the base member optionally with a vacuum maker and optionally with epithelial delaminating tools are examples of the described system or kit.

I claim as my invention:

1. A corneal stabilizer configured to be placed on an eye comprising: (a.) at least one outer radial surface adapted to contact the eye surface and cooperate with an inner radial surface to form a vacuum seal with that surface, (b.) the inner radial surface adapted to contact the eye surface upon deformation of the eye, but not to contact the eye without such deformation, (c.) an annular vacuum volume defined at the edges by the inner and outer radial surfaces, and (d.) an opening interior to the inner radial surface allowing extension of an interior corneal surface therethrough when the stabilizer is in contact with the eye with the inner and at least one outer radial surfaces.

2. The device of claim 1 further comprising a vacuum source.

3. The device of claim 1 further comprising a handle suitable for manipulation by a user.

4. The device of claim 1 further comprising a guide configured to couple with at least one supplemental device.

5. The device of claim 4 wherein the supplemental device comprises an epithelial delaminator.

6. The device of claim 1 further comprising a vacuum maker configured to close the opening through which the cornea is to be extended and to therefore deform the subject eye causing the corneal extension or protrusion.

7. The device of claim 1 wherein the inner radial surface is situated to be spaced from the subject eye when the outer radial surface contacts the subject eye without applying a vacuum.

8. The device of claim 7 wherein the inner radial surface is situated to be spaced from the subject eye at a distance of 0.0625+/−0.030 inches when the outer radial surface contacts the subject eye without applying a vacuum.

9. The device of claim 1 wherein the outer radial surface is at least partly compliant.

10. The device of claim 1 wherein the outer radial surface is substantially rigid.

11. The device of claim 1 wherein there is exactly one outer radial surface.

12. The device of claim 1 wherein there are more than one outer radial surfaces.

13. The device of claim 1 in combination with a vacuum maker adapted to close the open region.

14. The device of claim 5 and further comprising a lens.

15. The device of claim 5 wherein the epithelial delaminator is adapted to form an epithelial flap.

16. The device of claim 5 wherein the epithelial delaminator is adapted to form an epithelial pocket.

17. A method for stabilizing the cornea of a selected eye comprising the step of: a.) providing a stabilizing device according to claim 1, b.) providing vacuum to the annular vacuum volume, and c.) deforming the eye to cause the inner radial surface contact the eye and seal the stabilizing device against the eye, and to stabilize the cornea.

18. The method of claim 17 further comprising the step of separating at least a portion of the selected eye's epithelium from the cornea.

19. The method of claim 18 wherein the step of separating at least a portion of the epithelium includes forming a member selected from the group consisting of a separated epithelium, an epithilial flap having a hinge, and an epithelial pocket having one or more openings.

20. The method of claim 19 further including a step of performing a subtractive procedure upon the corneal surface and replacing the epithelium on that surface.

21. The method of claim 19 further comprising the step of introducing a lens onto the corneal surface and covering it with the separated epithilium.
